(19) United States
(12) Patent Application Publication
(10) Pub. No.: US 2023/0310417 A1
(43) Pub. Date: Oct. 5, 2023

(54) INJECTABLE ARIPIPRAZOLE FORMULATION

(71) Applicant: CIPLA LIMITED, Mumbai (IN)

(72) Inventors: Tushar RAJYAGURU, Mumbai (IN); Harshad HARDE, Mumbai (IN); Manoj WALUNJ, Mumbai (IN)

(21) Appl. No.: 18/020,898

(22) PCT Filed: Mar. 30, 2021
(86) PCT No.: PCT/IN2021/050323

§ 371 Date: Feb. 10, 2023

(30) Foreign Application Priority Data
Mar. 30, 2020 (IN) 202021013904

(51) Int. Cl. A61K 31/496; A61K 47/10; A61K 47/26; A61K 47/12; A61K 9/10

(52) U.S. Cl. CPC A61K 31/496; A61K 47/10; A61K 47/26; A61K 47/12; A61K 9/10; A61K 9/0019

(57) ABSTRACT

Stable ready-to-use extended release injectable pharmaceutical formulation comprising Aripiprazole are provided. The invention further relates to the method for preparing the above formulation, and a method for treating schizophrenia and related disorders employing the above formulation.

### CROSS REFERENCE TO RELATED APPLICATIONS

This application claims priority to co-pending Indian Provisional Patent Application Ser. No. 202021013904 filed on Mar. 30,2020. This application is incorporated herein by reference, in its entirety.

### FIELD OF INVENTION

The present invention relates to stable controlled release injectable pharmaceutical formulation comprising Aripiprazole. The formulation is in the form of ready-to-use formulations. The invention further relates to the method for preparing the above formulation, and a method for treating schizophrenia and related disorders employing the above formulation.

### BACKGROUND

Aripiprazole is a dopamine D₂ and Serotonin 5-HT_{1A} receptor agonist and antagonist of the Serotonin 5-HT_{2A} receptor. It has following structure:

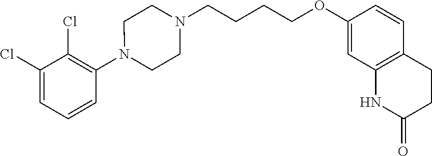 [SMILES: O=C1CCc2ccc(OCCCCN3CCN(c4cccc(Cl)c4Cl)CC3)cc2N1]

Aripiprazole is an atypical antipsychotic agent useful in treating schizophrenia. It has poor aqueous solubility (<1 μg/mL at room temperature).

Aripiprazole was first sold under the tradename Abilify as a tablet for oral administration. However, poor patient compliance and nonadherence to dosage regimen with such oral antipsychotics has been reported. In the treatment of schizophrenia, long-acting aripiprazole injectable formulation can simplify treatment process, improve patient's compliance and treatment adherence. It subsequently reduces the recurrence rate, improve patient's social interaction ability and quality of life. Ability Maintena has been approved by the FDA as a long-acting injectable formulation of Aripiprazole in 2013. Abilify Maintena is a lyophilized powder for reconstitution and need to be administered after reconstitution with diluent. Due to poor stability of deflocculated suspension of Abilify Maintena, the formulation is supplied as lyophilized powder. The single dose lyophilized formulation is associated with certain limitations such as the cumbersome and time consuming aseptic reconstitution process of dry powder, assurance of reconstitution, potential for dosing errors and wastage of remaining drug in vial after dose administration of reconstituted medication. The lyophilization process is also time consuming, incurs significant expense and complexity of equipment. Sterile diluent is needed to reconstitute Abilify Maintena to the required dose. Reconstituted products should be utilized as early as possible to render them sterile and avoid stability concern. Upon reconstitution with sterile diluent, Abilify Maintena forms deflocculated suspension. Deflocculated suspension have lower irritation potential at the site of administration. Although, keeping longer of this deflocculated suspension resulting in hard cake formation and hence difficult to re-disperse or re-suspend the drug particles.

Some attempts have been made to provide the controlled release aripiprazole injectable formulations. WO2005041937 discloses controlled release sterile freeze-dried aripiprazole formulation comprising of aripiprazole of a desired mean particle size and a vehicle therefor, which upon constitution with water and intramuscular injection releases aripiprazole over a period of at least about one week and up to about eight weeks.

US20190070171 discloses aripiprazole injectable suspension formulation comprising cellulose-based suspending agent and polyoxyethylene(20) sorbitan monooleate having prolonged shelf life.

US 20050032811 discloses pharmaceutical composition comprising aripiprazole and a carrier administered in a bolus injection resulted in an extended release profile similar to that obtained by the injection of a poly lactide-co-glycolide microsphere formulation containing the active agent.

However, all the prior attempts to produce alternative controlled release injection of Aripiprazole involve use of excipients which may interact with active and produce undesired side effect or are produced by tedious process of manufacture. Some formulations which are to be reconstituted before administration require a process of dilution leading to error in dosing. Some formulations are also not stable for longer time and difficult to re-disperse or re-suspend.

Thus, there exists a need for improved methods of delivering Aripiprazole, an antipsychotic drug to improve the patient compliance and maximize the pharmacological profile of the active agent.

The present invention overcomes the drawback that “particles would aggregate and settle from the aripiprazole suspension after long-term storage and hard to be redispersed,” and provides a method of prolonging shelf life of an aripiprazole injectable suspension formulation, as well as a stable injectable suspension formulation of aripiprazole having prolonged shelf life. Additionally, the present invention uses simplify and economical production process,

As Aripiprazole formulation has limited stability, development of long term storage stable Aripiprazole injection with lower irritational potential is very challenging. There are always chances of flocculation of particles when such products are stored for long time.

The present invention addresses the need for a pharmaceutically stable ready to use Aripiprazole formulation having long term storage stability, with regard to retaining the ready to use liquid dosage form, avoiding unacceptable degradation to undesired related substances and providing minimal or lower irritation at the site of administration. Also, since the formulations provided herein do not need the cumbersome procedure of reconstitution of dry powder, they are easy to administer and demonstrate reduction in dosing errors.

The inventors of present invention have provided ready-to-use extended release pharmaceutical injectable formulation of Aripiprazole having prolonged stability.

### OBJECT OF THE INVENTION

An object of the present invention is to provide long term storage stable ready-to-use Aripiprazole extended release injectable formulation.

Another object of the present invention is to provide long term storage stable ready-to-use extended release injectable formulation comprising Aripiprazole and pharmaceutically acceptable excipients.

Yet another object of the present invention is to provide a process of preparation of long term storage stable ready-to-use liquid injectable formulation.

Another object of present invention is to provide ready to use injectable formulation with minimal or non-irritation potential after administration at site.

In another object of the present invention, there is provided a method for treating patients suffering from schizophrenia.

### SUMMARY OF THE INVENTION

According to an aspect of present invention, there is provided long term storage stable ready-to-use Aripiprazole extended release injectable formulation. According to an aspect of present invention, there is provided long term storage stable ready-to-use liquid injectable formulation comprising Aripiprazole and pharmaceutically acceptable excipients. According to an aspect of present invention, there is provided a process of preparation of long term storage stable ready-to-use extended release injectable formulation. According to an aspect of present invention, there is provided a method for treating patients suffering from schizophrenia.

### DETAILED DESCRIPTION OF THE INVENTION

Maintaining the physical properties of drug substance with the help of suspending and surface active agents alone and/or in combination with pH adjusting agents and electrolytes, it is challenging to provide the formulation of present invention which is stable for prolonged period, easy for redispersion, have uniform dispersion of particles, exhibit the desired properties of good syringeability, resuspendability and injectability, have desired viscosity and low clogging. The benefits of extended release injectable suspension include but are not limited to
1) Reduce the dosing frequency, thus fewer injections are required.
2) Offer a better patient compliance especially for patients undergoing psychiatric treatment.
3) ability of therapeutically using active ingredients that are insoluble in conventional solvents,
4) decrease the incidence of side effects due to improvement in pharmacokinetic profile
5) increase the resistance of active ingredient to hydrolysis and oxidation,
6) exclusion of hepatic first-pass effect

Aripiprazole has poor water solubility and thus is difficult to formulate it as a stable injectable formulation. In accordance with the present invention, the stable aripiprazole formulation has been manufactured as an aqueous injection by using suspending agent and surface active agent in optimum amount. Without being bound to any theory, it has been believed that suspending agents and surfactants form layers around aripiprazole particles so that it remains in suspended form in the formulation and are easily redispersed on shaking. The inventors of present invention have done rigorous experimentation to choose the excipients such as suspending agent and surface active agent, electrolytes, pH adjusting agents and other excipients to provide the suspension of aripiprazole with desired and optimum suspension characteristics.

“Optimum suspension characteristics” means the most sediment volume measured indirectly through flocculation height (mm), and resuspendability in terms of the least number of strokes needed to re-disperse the flocculate. Redispersability is one of the major considerations in assessing the acceptability of a suspension. The sediment formed due to sitting should be easily re-dispersed by moderate shaking to yield a homogenous system. Measurement of the sedimentation volume and its ease of redispersion form two of the most basic evaluative procedures according to the text Theory and Practice of Industrial Pharmacy, L. Lachman et al., 2d Ed., pp 159, 180.

The ready to use pharmaceutical injectable formulation of present invention comprises of aripiprazole or its pharmaceutically acceptable salt thereof and pharmaceutically acceptable excipients thereof. The present invention provides ready to use long term storage stable pharmaceutical formulation comprising Aripiprazole or its pharmaceutically acceptable salts, isomers, racemates, enantiomers, hydrates, solvates, metabolites, polymorphs, and mixtures thereof.

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

To develop an effective and pharmaceutically acceptable injectable suspension, number of characteristics must be evaluated. These characteristics include resuspendability index, sedimentation height, syringeability, injectability, and viscosity. The inventors of present invention have provided formulation of aripiprazole or its pharmaceutically acceptable salt thereof with a combination of excipients which are present in optimum amount so that the formulation exhibits the desired characteristics of resuspendability, sediment height, syringeability, injectability, clogging, and viscosity at an optimum level.

Resuspendability describes the ability of the suspension to uniformly disperse with minimal shaking. Resuspendability can be a problem for suspensions that undergo “caking” upon standing due to settling of the deflocculated particles. “Caking” refers to a process by which the particles undergo growth and fusion to form a no dispersible mass of material. Sedimentation rate was assessed by observing the height in millimeters of the column of sedimentation visible in 20 millimeters of specimen suspension contained in a cylinder after shaking and then standing overnight. Larger heights were favorable indicating less separation with less supernatant liquid and less compaction of sedimentation.

Syringeability describes the ability of an injectable suspension to pass easily through a hypodermic needle on transfer from a syringe prior to injection. It includes characteristics such as ease of withdrawal, clogging and foaming tendencies, and accuracy of dose measurements. The increase in the viscosity, density, particle size, and concentration of solids in suspension hinders the syringeability of suspensions.

The injectability refers to the performance of the suspension during injection. Injectability includes factors such as pressure or force required for injection, evenness of flow, aspiration qualities, and freedom from clogging.

Viscosity describes the resistance that a liquid system offers to flow when it is subjected to an applied shear stress. A more viscous system requires greater force or stress to make it flow at the same rate as a less viscous system. A liquid system will exhibit either Newtonian or non-Newtonian flow based on a linear or a non-linear increase, respectively, in the rate of shear with the shearing stress. Structured vehicles used in suspensions exhibit non-Newtonian flow and are typically plastic, pseudoplastic, or shear-thinning with some thixotropy (exhibiting a decrease in viscosity with an increase in the rate of shear).

The Aripiprazole injectable formulation of the present invention may include pharmaceutically acceptable salts of aripiprazole such as, but not limited to, those formed by using acids such as maleic acid, fumaric acid, succinic acid, malic acid, tartaric acid, malonic acid, oxalic acid, or phthalic acid oxalic acid, tartaric acid or succinic acid , camphorsulfonic acid and phosphoric acid. The present invention comprises of anhydrous or monohydrate salt forms of aripiprazole or an admixture containing both. If the monohydrate is used, the maintenance of an extended drug plasma concentration is possible. The preferable salt of Aripiprazole used in formulation of present invention is aripiprazole monohydrate which has a chemical name of 7-[4-[4-(2,3-dichlorophenyl)-1-piperazinyl]butoxy]-3,4 dihydrocarbostyril monohydrate.

In an embodiment, the present invention provides an extended release formulation of aripiprazole comprising aripiprazole monohydrate suspended in a pharmaceutically acceptable vehicle with pharmaceutically acceptable excipients.

The pharmaceutically acceptable excipients included in formulation of present invention include, not limited to, suspending agent, viscosity modifiers, water insoluble co-surfactant, water soluble surfactant, buffer system, and aqueous vehicle and the like.

The term “suspending agent” as used herein refers to a pharmaceutical acceptable excipient that promotes particle suspension or dispersion and reduces sedimentation. Suspending agents retard settling and agglomeration of particles by minimizing interparticle attraction. Suspending agents include protective colloids and viscosity-inducing agents. Protective colloids differ from surfactants in that they do not reduce interfacial tension. Many agents that are protective colloids in low concentration (<0.1%) are viscosity builders in higher concentrations (>0.1%). The increase in viscosity of the solution is helpful to prevent sedimentation of the suspended particles. A suspension has well developed thixotropy. At rest the formulation is sufficient viscous to prevent sedimentation and thus aggregation or caking of the particles. When agitation is applied, the viscosity is reduced and provide good flow characteristic.

Examples of suspending agents include polysaccharides, inorganic salts, and polymers. Specific examples of suspending agents include, without limitation, alginates, colloidal silicon dioxide, agar, calcium stearate, magnesium aluminium silicate, guar gum, acacia, tragacanth, xanthan gum, bentonite, carbomer, carageenan, gelatin, polyethylene glycol, povidone, dextrin, medium-chain triglycerides, sucrose, chistosan, polyoxyethylene, polyoxy-propylene ethers and combinations thereof. The concentration of the suspending agent can generally be from about 0.1 mg/mL to about 200 mg/mL, or from about 0.5 mg/mL to about 100 mg/mL. In preferred embodiment, concentration of the suspending agent is from about 1 mg/mL to about 90 mg/mL. The formulation of present invention does not contain cellulose derivatives such as carboxymethylcellulose sodium (Na-CMC) as the suspending agent. During the experimentation, to arrive at the formulation of present invention, it was observed by the inventors that the anions produced in the solution after the dissolution of Na-CMC in water might interact with each other as well as with water/excipients molecule through electrostatic forces and exhibit considerable electrostatic interacting force via hydrogen bonding. These electrostatic interaction attribute to strong binding between Na-CMC, Aripiprazole particle surface and water molecules. Therefore, it was observed that the viscosity generation in formulation using Na-CMC was very high. It resulted in reduction of sedimentation rate, but eventually had negative effect on resuspendability of suspension. A very low Na-CMC concentration of 0.1% w/v was also unable to improve resuspendability of suspension.

In a preferred embodiment, the suspending agent is selected from polyethylene glycol (e.g., polyethylene glycol 3350, polyethylene glycol 4000, polyethylene glycol 6000), povidone, and combinations thereof. In preferred embodiment, the suspending agent is polyethylene glycol 4000. For polyethylene glycol 4000, the concentration can be from about 10 mg/mL to about 100 mg/mL, from about 20 mg/mL to about 90 mg/mL, from about 30 mg/mL to about 80 mg/mL. The PEG 4000 has dual action of viscosity modifier as well as wetting agent. The viscosity modification by suspending agent in suspension was helpful to reduce the particle interaction as well as decrease the settling time. In absence of PEG 4000, the suspension have formed the compact cake with short sediment height, and it had poor impact on resuspendability.

The term “surfactant” as used herein means agents that lower the surface tension (or interfacial tension) between two liquids or between a liquid and a solid. Surfactants are usually organic compounds that are amphiphilic, i.e., they contain both hydrophobic groups (tails) and hydrophilic groups (heads). Therefore, a surfactant contains both a water-insoluble (or oil-soluble) component and a water-soluble component.

Surfactants can be classified according to polar head group. A non-ionic surfactant has no charged groups in its head. Nonionic surfactants have covalently bonded oxygen-containing hydrophilic groups, which are bonded to hydrophobic parent structures. The head of an ionic surfactant carries a net positive, or negative charge. If the charge is negative, the surfactant is more specifically called anionic; if the charge is positive, it is called cationic. If a surfactant contains a head with two oppositely charged groups, it is termed zwitterionic. An ideal interaction of hydrophilic and lipophilic group of surfactant with particle surface could be reason for obtaining appropriate suspension stability.

In the presence of a suspending agent, the addition of a surfactant can further enhance the resuspendability and sediment height of the formulation. In one embodiment of present invention, provided is a formulation comprising aripiprazole or salt thereof, suspending agent and a surfactant. The surfactant can be a non-ionic surfactant, an ionic surfactant (anionic or cationic), or a zwitterionic surfactant. The surfactant in suspensions reduce the interfacial tension between the solid particles and the liquid vehicle. The powder then readily wetted by the liquid vehicle leads to formation of flocculated systems by dispersion. Examples of surfactants include polysorbate (such as polysorbate 80 and Polysorbate 20), sorbitan ester, polyoxyethylene hydrogenated castor oil (such as polyoxyethylene hydrogenated castor oil 50 and polyoxyethylene hydrogenated oil castor oil 60), poloxamer 188 or polyoxyethylene castor oil. Preferred surfactants include Polysorbate 20, Polysorbate 80, sorbitan monolaurate, and poloxamer 188. In one embodiment, the surfactant is polysorbate, such as polysorbate 20 or polysorbate 80. In yet another preferred embodiment, the surfactant is sorbitan monolaurate. The concentration of surfactant can be from about 0.1 mg/mL to about 50 mg/mL, from about 0.5 mg/mL to about 30 mg/mL, from about 0.5 mg/mL to about 20 mg/mL, or from about 0.5 mg/mL to about 10 mg/mL. In preferred embodiments, the surfactant is present in an amount of about 0.2% to about 2%. In embodiments where the surfactant is polysorbate 20, it is preferably used at concentrations from about 0.1% (w/w) to about 2.0% (w/w), and in some case in concentrations from about 1% (w/w) to about 2% (w/w), from about from about 0.1% (w/w) to about 1.5% (w/w), from about 0.1% (w/w) to about 1.0% (w/w), from about from about 0.5% (w/w) to about 1.5% (w/w), or from about from about 0.5% (w/w) to about 1% (w/w).The inclusion of surfactants along with suspending agent have helped the formulation to form the flocculated suspension, which was easily resuspendable. The formulation of present invention expressed “controlled flocculation”. It was observed by inventors that balancing between suspending agents, surfactant, and ionic species were helpful to obtain stabilized suspension formulation of present invention.

The extended release formulations of the present invention comprises polysorbate 20 as the surfactant, The extended release formulations of the present invention comprises poloxamer 188 as the surfactant, it is preferably used at a concentration of from about 0.1% (w/w) to about 1% (w/w).

The extended release suspension formulation of present invention also includes a buffering and/or isotonic agents. Examples of buffering agents include, without limitation, sodium dihydrogenphosphate, disodium hydrogenphosphate, sodium phosphate, potassium dihydrogenphosphate, dipotassium hydrogenphosphate, citric acid, sodium hydroxide, hydrochloric acid or the combinations thereof. It was suggested that buffering agents being soluble electrolyte formed electrical double layer surrounding each particle, thus additionally helps in flocculation. The Aripiprazole preferably as aripiprazole monohydrate base is present to reduce particle agglomeration in the formulation of present invention. The electrolyte concentration more than 80 mM was necessary to have electrostatic stabilization of suspension.

It is important to note here that aqueous solution of Aripiprazole or its pharmaceutically acceptable salts at higher pH (3.0 to 9.0) remains stable without any physical instability. The formulation of present invention is a ready-to-use formulation that is stable at room temperature as well as at lower temperatures between 2° to 8° C.

The particle size is a critical attribute for in-vitro and in-vivo release rate. In case of Aripiprazole suspension, the particle size plays an important role in controlling the in vivo release rate from extended release formulation of present invention. In order to provide a formulation providing extended release of aripiprazole or its pharmaceutically acceptable salt thereof over at least about a 28-day period, the mean particle size should be from about 1 μm to about 100 μm. In some embodiments, the mean particle size of the crystals of Aripiprazole or a pharmaceutically acceptable salt thereof is from about 1 μm to about 60 μm, In some embodiments, the mean particle size of the crystals of Aripiprazole or a pharmaceutically acceptable salt thereof is at least about, 1 μm , 5 μm, 10 μm 20 μm, 30 μm, 40 μm, 50 μm, 60 μm.

In some embodiments, the formulation contains at least 200 mg/mL aripiprazole or a pharmaceutically acceptable salt thereof. In some embodiments, the concentration is at least 100 mg/mL, 150 mg/mL, 230 mg/mL, 240 mg/mL, 250 mg/mL, 260 mg/mL, 270 mg/mL, 280 mg/mL, 290 mg/mL, 300 mg/mL, 310 mg/mL, 320 mg/mL, 330 mg/mL, 340 mg/mL, 350 mg/mL, 360 mg/mL, 370 mg/mL, 380 mg/mL, 390 mg/mL, 400 mg/mL, 410 mg/mL, or 420 mg/mL of aripiprazole or its salt. Preferably, the concentration of aripiprazole monohydrate in the formulation of present invention is equal to 200 mg/ml.

In a preferred embodiment, the formulation of present invention comprises of aripiprazole monohydrate , the suspending agent as PEG 4000 at about 5% (w/w), the surfactant as polysorbate 20 at about 1% (w/w) to about 2%, in a phosphate buffer (0.6% sodium dihydrogen phosphate monohydrate and 0.112% sodium hydroxide aqueous solution) with aripiprazole with a D90 of about 1-100 μm.

In a preferred, the formulation of present invention is not a freeze dried composition of aripiprazole or its pharmaceutically acceptable salt.

The physical properties critically impacted the release behaviour of the drug product. Amorphous and crystalline nature of drug substance also play a major role for the suspension and colloidal solutions. Crystallinity of the drug substance i.e. Aripiprazole governs dissolution and in vivo release rate from extended release formulation of present invention. Preferably, Aripiprazole in the formulation of present invention is of crystalline or amorphous or combination thereof in nature. The formulations of the present invention can be packaged in any suitable sterile vial or prefilled syringe or container fit for the sterile storage of a pharmaceuticals. The formulation of present invention can be provided in a kit or package that includes a container enclosing the formulation. Suitable containers can be glass vials, i.e. Schott treated vials, molded glass vials, and CZ resin vials, polypropylene or polyethylene vials or other special purpose containers. Suitable containers can be prefilled syringe such as glass prefilled syringes, plastic prefilled syringes. Containers are of a size sufficient to hold one or more doses of aripiprazole. The container may be part of a syringe or separate from the syringe. The kit or package also includes a needle that can be suitably mounted to the syringe. The size of the needle, in some embodiments, is equal to or smaller than 18 G, 19 G, 20 G, 21 G, 22 G, 23 G, 24 G, or 25 G. In one embodiment, the needle has a size that is 20 G or smaller. In one embodiment, the needle has a size that is 21 G or smaller. In one embodiment, the needle has a size that is 22 G or smaller. In one embodiment, the needle has a size that is 23 G or smaller.

The term “stable formulations” refers that aripiprazole ready-to-use injection formulations of present invention are physically as well as chemically stable as demonstrated by compliance to acceptable specification when the formulation is stored at convenient temperature, such as between about 0° C. and about 60° C., for a commercially reasonable period of time, such as at least about 1 day, at least about 1 week, at least about 1 month, at least about 3 months, at least about 6 months, at least about 1 year, or at least about 2 years. For purposes of the present invention, “long term storage’ shall be understood to include at least time periods which are in excess of those observed when currently available lyophilized aripiprazole formulations are reconstituted. Suitably, the suspension of aripiprazole of present invention remains physically stable, with no precipitation or crystallization or color change upon storage and the shelf life period of 18-24 months when stored at room temperature. Suitably, the suspension of aripiprazole remains chemically stable when stored at room temperature (about 25° C.) and at refrigerated conditions (2-8° C.), wherein various parameters such as the drug content (assay of Aripiprazole) and content of related substances, i.e. known and unknown impurities remains within specified limits such as those specified according to ICH guidelines, upon storage for prolonged period of time such as for at least 12 months, preferably for 18 months, more preferably 24 months or longer.

The formulation of present invention is substantially free of impurities. For purposes of the present invention, “substantially free of impurities’ shall be understood to include aripiprazole containing formulations in which the amount of total impurities is less than about 5% of the sum of peak areas of all degradants, as calculated on a normalized peak area response (“PAR) basis as determined by high performance liquid chromatograph (“HPLC) after a period of about 18 months at a temperature of from about 15° C. to about 25° C. The amount of impurities is further calculated as being based upon the original amount of aripiprazole (or salt thereof) being present in the composition or formulation. Preferably, the said stable formulations of aripiprazole prevent degradation of aripiprazole such that not more than 2% , not more than 1% , not more than 0.4%, not more than 0.2% of aripiprazole impurity or impurities are formed over the storage period. In yet another preferred embodiment the value of assay of aripiprazole remains within the specified limit of 90-110% by weight of the label claim; the highest unknown impurity remains within the specified limit of not more than 0.2%; the known Impurities A, B, C and D remains within the specified limit of not more than 0.29% and the Impurity A remains within the specified limit of not more than 1.0%. The total impurities remain below 2.0%, preferably below 1.0%.

In some preferred aspects of the invention, the time for which long term storage are contemplated include periods of at least about 24 months or longer with such that the formulation is substantially free of impurities when stored at room temperature. While not wishing to be bound by any theory whatsoever, it is believed that the use of excipients such as viscosity modifying agents, surfactants, buffers and isotonicity agents in preparing aripiprazole compositions of the invention play a significant role in reducing the degradation of aripiprazole thereby prolonging the shelf-life of said aripiprazole formulations.

The addition of the components of the injection for the preparation of injection can be achieved by methods known in the art. For example, one or more of the components may be added to each other and then into a common receptacle for mixing, or the components may be added to a common receptacle in a particular order, or the components may be added to a common receptacle simultaneously. In preferred embodiment, the aqueous solution of all the pharmaceutically acceptable excipients such as suspending agent, surfactant, viscosity modifier, buffer is prepared. Measured quantity of aripiprazole salt is added gradually to prepared aqueous solution of excipients under continuous stirring. The mixture is stirred to ensure uniform mixing of aripiprazole in aqueous solution. The pH of suspension measured and if required adjusted to 3.0-9.0 using hydrochloric acid/sodium hydroxide solution. Total volume of suspension was adjusted using WFI and stirred to ensure uniform mixing. Final pH of suspension measured and suspension filled in PFS. In yet another preferred embodiment, the aqueous solution of all the pharmaceutically acceptable excipients such as suspending agent, surfactant, viscosity modifier, buffer is prepared to which measured quantity of aripiprazole was added under continuous stirring to bring uniformity in suspension. This homogeneous suspension was milled suitable milling technique such as or high pressure homogenizer or media mill or wet mill or microfluidizer to achieve desired particle size. The pH of milled suspension was measured and if required adjusted to 3.0-9.0 using hydrochloric acid/sodium hydroxide solution. Total volume of suspension was adjusted using WFI and stirred to ensure uniform mixing. Final pH of suspension measured and suspension filled in PFS.

The ICH storage stability studies were performed on ready to use aripiprazole suspension packaged in the proposed commercial primary packaging and closure system. The stability study samples were stored at 25° C./65% RH and accelerated storage conditions were 40° C./75% RH. Up to 6 months stability data have been provided. The necessary parameters viz., assay, related substances, particle size distribution, pH, osmolality, resuspendability time, sediment height, and dissolution were tested and found to be within specification at both stability conditions. The stability data suggested long term stability at room temperature. The present invention further provides methods of treating a patient suffering from a psychotic disease or condition, such as schizophrenia or bipolar depression by intramuscular injection of the formulations of the present invention at a recommended dose of 200-400 mg monthly.

### EXAMPLES

The following examples are for the purpose of illustration of the invention only and are not intended to limit the scope of the present invention in any manner whatsoever.

### Example 1: Aripiprazole ready to use injection, 200 mg/mL

TABLE 1

| Ingredients | Quantity (mg/mL) |
|---|---|
| Aripiprazole Monohydrate | 200 |
| Polyethylene Glycol 4000 | 10 to 100 |
| Sorbitan Monolaurate (SPAN 20) | 0.1 to 3.0 |
| Polysorbate 20 (Tween 20) | 1 to 20 |
| Citric acid monohydrate | 1 to 15 |
| Sodium dihydrogen phosphate monohydrate | 2 to 20 |
| Sodium Hydroxide/HCL | QS to pH |
| Water for injection | QS to 1 mL |
| pH range | 3.0 to 9.0 |

Process of preparation: The aqueous solution of containing excipients such as polyethylene glycol 4000, Polysorbate 20, sorbitan laureate, citric acid monohydrate and sodium dihydrogen phosphate monohydrate were dissolved completely in the water for injection (WFI) to prepare the aqueous phase. A required quantity of aripiprazole monohydrate was added gradually into prepared aqueous solution under continuous stirring to ensure uniform mixing. The pH of suspension measured and if required adjusted using hydrochloric acid/sodium hydroxide solution. Total volume of suspension was adjusted using WFI and stirred to ensure uniform mixing. Final pH of suspension measured and suspension filled in PFS. The suspension was milled using media mill to get desired particle size.

Stability studies: The stability studies and results of formulation of example 1 are shown in Table 2. The stability studies of ready-to-use aqueous Aripiprazole composition mentioned according to example 1 demonstrated that the redispersion time, assay, and impurities levels after real time and accelerated studies over 6 months were within the acceptable limits.

TABLE 2

| | | | 25° C./60% RH | | | 40° C./20% RH | | |
|---|---|---|---|---|---|---|---|---|
| Parameter | | Initial | 1 M | 3 M | 6 M | 1 M | 3 M | 6 M |
| Osmolarity | | 286 | 254 | — | 261 | 253 | — | 277 |
| Redispersion | | NP* | NP* | NP* | 10 | NP* | NP* | 20 |
| time (sec) | | | | | | | | |
| % sediment | | NP* | NP* | NP* | 81.6 | NP* | NP* | 86.2 |
| Assay (%) | | 103.5 | 104.1 | 104.4 | 104 | 103.1 | 103.6 | 104 |
| Impurity | SMI | ≤0.10% | ≤0.10% | ≤0.10% | 0.26% | ≤0.10% | ≤0.10% | 0.29% |
| levels | Total | ≤0.10% | ≤0.10% | ≤0.10% | 0.26% | ≤0.10% | ≤0.10% | 0.44% |
| PSD | D90 | 11.54 | 12.07 | 12.37 | 12.07 | 14.06 | 13.23 | 14.29 |

NP: Not Performed

### Example 2: Aripiprazole ready to use injection, 200 mg/mL

TABLE 3

| Ingredients | Quantity (mg/mL) |
|---|---|
| Aripiprazole Monohydrate | 200 |
| Polyethylene Glycol 4000 | 10 to 100 |
| Polysorbate 20 (Tween 20) | 5 to 30 |
| Citric acid monohydrate | 1 to 15 |
| Sodium dihydrogen phosphate monohydrate | 2 to 20 |
| Sodium Hydroxide/HCL | QS to pH |
| Water for injection | QS to 1 mL |
| pH range | 3.0 to 7.0 |

Process of preparation: The aqueous solution of containing excipients such as polyethylene glycol 4000, polysorbate 20, citric acid monohydrate and sodium dihydrogen phosphate monohydrate were dissolved completely in the water for injection (WFI) to prepare the aqueous phase. Required quantity of aripiprazole monohydrate was added gradually into prepared aqueous solution under continuous stirring. The mixture stirred to ensure uniform mixing of aripiprazole in aqueous solution to prepare uniform suspension. The pH of suspension measured and if required adjusted using hydrochloric acid/sodium hydroxide solution. Total volume of suspension was adjusted using WFI and stirred to ensure uniform mixing. Final pH of suspension measured and suspension filled in PFS

Stability studies: The stability studies and results of formulation of example 2 are shown in Table 4. The stability studies of ready-to-use aqueous Aripiprazole composition mentioned according to example 2 demonstrated that the redispersion time, assay, and impurities levels after real time and accelerated studies over 3 months were within the acceptable limits.

TABLE 4

| | | | 25 C./60% RH | | 40 C./20% RH | |
|---|---|---|---|---|---|---|
| Parameter | | Initial | 1 M | 3 M | 1 M | 3 M |
| Osmolarity | | 212 | 218 | NP* | 218 | NP* |
| Redispersion time | | NP* | 20 | 20 | 20 | 20 |
| (sec) | | | | | | |
| % Sediment height | | NP* | 57.1 | 42.92 | 53.3 | 54.14 |
| Assay | % LA | 95.9 | 96.3 | 94.7 | 95.5 | 97 |
| Impurity | SMI | ≤0.10% | ≤0.10% | ≤0.10% | ≤0.10% | ≤0.10% |
| levels | Total | ≤0.10% | ≤0.10% | ≤0.10% | ≤0.10% | ≤0.10% |
| PSD | D90 | 40.76 | 39.34 | 36.89 | 42.93 | 43.24 |

NP: Not Performed

### Example 3: Aripiprazole ready to use injection, 200 mg/mL

TABLE 5

| Ingredients | Quantity (mg/mL) |
|---|---|
| Aripiprazole Monohydrate | 200 |
| Polyethylene Glycol 3350 | 10 to 100 |
| Sorbitan monolaurate | 0.1 to 3.0 |
| Polysorbate 20 | 1 to 20 |
| Citric acid monohydrate | 1 to 15 |
| Sodium dihydrogen phosphate monohydrate | 2 to 20 |
| Sodium Hydroxide/HCL | QS to pH |
| Water for injection | QS to 1 mL |
| pH range | 3.0 to 7.0 |

Process of preparation: The aqueous solution of containing excipients such as polyethylene glycol 3350, polysorbate 20, citric acid monohydrate and sodium dihydrogen phosphate monohydrate were dissolved completely in the water for injection (WFI) to prepare the aqueous phase. Required quantity of aripiprazole monohydrate was added gradually into prepared aqueous solution under continuous stirring. The mixture stirred to ensure uniform mixing of aripiprazole in aqueous solution to prepare uniform suspension. The pH of suspension measured and if required adjusted using hydrochloric acid/sodium hydroxide solution. Total volume of suspension was adjusted using WFI and stirred to ensure uniform mixing. Final pH of suspension measured and suspension filled in PFS.

### Example 4: Aripiprazole ready to use injection, 200 mg/mL

TABLE 6

| Ingredients | Quantity (mg/mL) |
|---|---|
| Aripiprazole Monohydrate | 200 |
| Polyethylene Glycol 4000 | 10 to 100 |
| Polysorbate 20 | 5 to 30 |
| Sodium dihydrogen phosphate monohydrate | 1 to 5 |
| Sodium chloride | 3 to 9 |
| Sodium Hydroxide/HCL | QS to pH |
| Water for injection | QS to 1 mL |
| pH range | 3.0 to 9.0 |

Process of preparation:The aqueous solution of containing excipients such as polyethylene glycol 4000, polysorbate 20, sodium dihydrogen phosphate monohydrate and sodium chloride were dissolved completely in the water for injection (WFI) to prepare the aqueous phase. Required quantity of aripiprazole monohydrate was added gradually into prepared aqueous solution under continuous stirring. The mixture stirred to ensure uniform mixing of aripiprazole in aqueous solution to prepare uniform suspension. The pH of suspension measured and if required adjusted using hydrochloric acid/sodium hydroxide solution. Total volume of suspension was adjusted using WFI and stirred to ensure uniform mixing. Final pH of suspension measured and suspension filled in PFS.

### Example 5: Aripiprazole ready to use injection, 200 mg/mL

TABLE 7

| Ingredients | Quantity (mg/mL) |
|---|---|
| Aripiprazole Monohydrate | 200 |
| Polyethylene Glycol 4000 | 10 to 100 |
| Sorbitan monolaurate | 0.1 to 3.0 |
| Polysorbate 20 | 1 to 20 |
| Glacial acetic acid | 0.5 to 5.0 |
| Sodium acetate | 2.5 to 15.0 |
| Water for injection | QS to 1 mL |
| pH range | 3.0 to 7.0 |

Process of preparation: The aqueous solution of containing excipients such as PEG 4000, sorbitan monolaurate, polysorbate 20, acetic acid and sodium acetate were dissolved completely in the water for injection (WFI) to prepare the aqueous phase. Required quantity of aripiprazole monohydrate was added gradually into prepared aqueous solution under continuous stirring. The mixture stirred for 2 hours using overhead stirring to ensure uniform mixing of aripiprazole in aqueous solution to prepare uniform suspension. The pH of suspension measured and volume of suspension was adjusted using WFI. The suspension again stirred to ensure uniform mixing. Final pH of suspension measured and suspension filled in PFS.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to a “cosolvent” refers to a single cosolvent or to combinations of two or more cosolvents, and the like.

We claim,

1. A stable ready-to-use extended release formulation, comprising
i) aripiprazole or a pharmaceutically acceptable salt thereof, and
ii) pharmaceutical acceptable excipients thereof when stored for 6 months in a sealed, sterile container at 60% RH at a temperature from 25° C., contains no more than 1.0% total impurity, as measured by HPLC.

2. The formulation as claimed in claim 1, wherein the pharmaceutically acceptable salt thereof is aripiprazole monohydrate.

3. The formulation as claimed in claim 1, wherein the concentration of aripiprazole monohydrate is about 200 mg/mL of total formulation.

4. The formulation as claimed in claim 1, wherein the particle size of aripiprazole monohydrate from about 10 μm. to about 100 μm.

5. The formulation of claim 1 wherein the formulation is in the form of a suspension.

6. The formulation as claimed in claim 1, wherein the pharmaceutically acceptable excipients are suspending agent, surfactant, buffer system, and aqueous vehicle.

7. The formulation of claim 6, wherein the suspending agents comprises alginates, colloidal silicon dioxide, agar, calcium stearate, magnesium aluminium silicate, guar gum, acacia, tragacanth, xanthan gum, bentonite, carbomer, carageenan, gelatin, polyethylene glycol, povidone, dextrin, medium-chain triglycerides, sucrose, chistosan, polyoxyethylene, polyoxy-propylene ethers and combinations thereof

8. The formulation of claim 7, wherein the suspending agent selected from polyethylene glycol 3350, polyethylene glycol 4000, polyethylene glycol 6000, povidone, and combinations thereof is present in an amount of from about 10 mg/mL to about 100 mg/mL of total formulation.

9. The formulation of claim 6, wherein the surfactants comprises polysorbate 80, Polysorbate 20, sorbitan ester, polyoxyethylene hydrogenated castor oil, poloxamer 188.

10. The formulation of claim 9, wherein the surfactant selected from Polysorbate 20, Polysorbate 80, sorbitan monolaurate and combinations thereof is present in an amount of from about 0.1 mg/mL to about 30 mg/mL of total formulation.

11. The formulation of claim 6, wherein the buffer comprises of sodium dihydrogenphosphate, disodium hydrogenphosphate, sodium phosphate, potassium dihydrogenphosphate, dipotassium hydrogenphosphate, citric acid, sodium hydroxide, hydrochloric acid or the combinations thereof

12. The formulation of claim 1, wherein the pH of formulation is from 3.0 to 9.0.

13. The formulation of claim 12, wherein the pH of formulation is from 3.0 to 7.0.

14. The formulation of claim 1, wherein the vehicle comprises of water for injection.

15. The formulation of claim 1, wherein the formulation provides extended release of aripiprazole or its pharmaceutically acceptable salt thereof over at least about a 28-day period.

16. A method of treating schizophrenia, comprising administering to a patient in need thereof ready-to-use formulation comprising aripiprazole or a pharmaceutically acceptable salt thereof, and pharmaceutically acceptable excipients thereof, wherein the composition, when stored for six months in a sealed, sterile vial at 60% RH at a temperature from 25° C., contains no more than 1.0% total impurity, as measured by HPLC.

17. The method according to claim 16, wherein the composition is ready-to-use and is directly injected into the patient.

18. A process for the preparation of stable pharmaceutical preparation of aripiprazole or its pharmaceutically acceptable salts in a ready-to-use form, comprising the steps of:
a) dissolving suspending agent, surfactant, buffer in a solvent and obtaining a solution,
b) dissolving aripiprazole or its pharmaceutically acceptable salts in solution obtained in step a) by stirring and thus obtaining the suspension;
c) adjusting the pH of the composition between 3.0 to 9.0;
d) filling the product suspension in suitable containers/closures to obtain a preparation in a ready-to-use form; and
e) optionally sparging with inert gas any time during the process for the preparation.
